(19) United States
(12) Patent Application Publication
(10) Pub. No.: US 2021/0016029 A1
(43) Pub. Date: Jan. 21, 2021

(54) PORTABLE VENTILATOR AND METHODS FOR PROVIDING OSCILLATORY FLOW

(71) Applicants: MacKay Memorial Hospital, Taipei City (TW); National Chiao Tung University, Hsinchu City (TW)

(72) Inventors: Hsu-Tah KUO, Taipei City (TW); Chien-Liang WU, Taipei City (TW); Wen-Jui WU, Chiayi City (TW); Yu-Te LIAO, Hsinchu City (TW); Shao-Yung LU, Hsinchu City (TW)

(73) Assignees: MacKay Memorial Hospital, Taipei City (TW); National Chiao Tung University, Hsinchu City (TW)

(21) Appl. No.: 16/516,267

(22) Filed: Jul. 19, 2019

(51) Int. Cl. A61M 16/00; A61M 16/12; A61M 16/20

(52) U.S. Cl. CPC A61M 16/0006; A61M 16/125; A61M 16/208; A61M 16/203; A61M 2202/0208; A61M 2205/3334; A61M 2230/40; A61M 2205/50; A61M 2016/0027

(57) ABSTRACT

Disclosed herein is a portable ventilator and a method for providing an oscillatory flow to a subject in need thereof. The method comprises: (a) forming a gas mixture comprising pure oxygen and air; (b) converting the gas mixture into the oscillatory flow by applying thereto a predetermined oscillatory frequency and a predetermined ventilatory duration; (c) outputting the oscillatory flow of the step (b) at a first jet pressure, in which the outputted oscillatory flow has a first flow rate; and (d) modulating the outputted oscillatory flow of the step (c) by, (i) varying the respective amounts of the pure oxygen and the air in the gas mixture; or (ii) varying the predetermined ventilatory duration of the step (b), in which if the fist jet pressure is smaller than the predetermined jet pressure, then decreases the predetermined ventilatory duration; or if the first jet pressure is greater than the predetermined jet pressure, then increases the predetermined ventilatory duration.

### BACKGROUND OF THE INVENTION

### 1. Field of the Invention

The present disclosure in general relates to the field of artificial respiration. More particularly, the present disclosure relates to methods for regulating an artificial respiration apparatus to provide high-frequency ventilation to a subject in need thereof.

### 2. Description of Related Art

To avoid unnecessary waste and to increase cost-efficiency in health care to the ventilator-dependent patients are the main purpose of the medical related field. In practice, though various conventional mechanical ventilators can be applied to those in need, these instruments often cause complications including pulmonary pressure injury, over expansion of alveoli, and high airway pressure in long-term treatment. To cure such defects, high-frequency ventilator capable of providing low-volume and high flow rate of air flow to the patients has been constructed and proved to be effective in reducing lung injury thereby reducing mortality and complications caused by airway/lung pressure problems. Accordingly, the high-frequency ventilator has become widely-used, particularly in pediatric clinics to prevent pulmonary damage, respiratory arrest and/or chronic lung disease in premature infants.

Despite the advantages the high-frequency ventilator may possess, yet there are still some side effects on certain circumstances. In the case of discharging by only passive exhalation, the higher inhaling flow rate is given, the higher average pulmonary pressure occurs. If the lungs hardly exhale completely, the patient suffers an abnormal retention of air in the lungs, which greatly reduce the ventilation efficiency. Further, the present instruments for producing high-frequency ventilation are bulky and costly therefore only usable in medical institutions, which does not meet the requirement of home care and hospice.

In view of the foregoing, there exists in the related art a need for a portable apparatus and an improved method for regulating the apparatus to provide high-frequency ventilation to a subject in need thereof.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, the present disclosure aims at providing a portable apparatus and an improved method of regulating the apparatus to provide high-frequency ventilation to a subject in need thereof while eliminating any possible lung damage.

Accordingly, one aspect of the present disclosure is directed to a method for providing an oscillatory flow to a subject in need thereof. The method comprises: (a) forming a gas mixture comprising pure oxygen and air; (b) converting the gas mixture into the oscillatory flow by applying thereto a predetermined oscillatory frequency and a predetermined ventilatory duration; (c) outputting the oscillatory flow of the step (b) at a first jet pressure, in which the outputted oscillatory flow has a first flow rate; and (d) modulating the outputted oscillatory flow of the step (c) by, (i) respectively matching the first flow rate and the first jet pressure with a predetermined flow rate or a predetermined jet pressure by varying the respective amounts of the pure oxygen and the air in the gas mixture; or (ii) matching the first jet pressure with the predetermined jet pressure by varying the predetermined ventilatory duration of the step (b), in which if the fist jet pressure is smaller than the predetermined jet pressure, then decreases the predetermined ventilatory duration; or if the first jet pressure is greater than the predetermined jet pressure, then increases the predetermined ventilatory duration.

According to some embodiments of the present disclosure, in the step (a), the pure oxygen and the air are independently suppled from their sources and mixed in a reservoir having a constant volume to form the gas mixture.

According to some embodiments of the present disclosure, in the step (c), the first jet pressure of the oscillatory flow is in proportional to the amount of the gas mixture in the reservoir.

According to some embodiments of the present disclosure, in the step (d)(i), if the first flow rate is smaller than the predetermined flow rate, then increases the respective amounts of the pure oxygen and the air in the gas mixture; or if the first flow rate is greater than the predetermined flow rate, then decreases the respective amounts of the pure oxygen and the air in the gas mixture.

In certain examples of the present disclosure, the predetermined flow rate is about 0 L/min to 30 L/min.

According to some embodiments of the present disclosure, the gas mixture of the step (a) has a predetermined oxygen concentration.

In some optional embodiments, the present method further comprises (e) detecting an actual oxygen concentration in the gas mixture in the step (c) and/or after the step (d)(i); and (f) matching the actual oxygen concentration with the predetermined oxygen concentration by varying the amount of the pure oxygen in the gas mixture of the step (a).

In certain embodiments, the predetermined oxygen concentration is about 20 vol % to 90 vol %.

According to certain embodiments of the present disclosure, the predetermined oscillatory frequency is about 1 Hz to 8 Hz.

According to certain embodiments of the present disclosure, the predetermined jet pressure is about 5 psi to 45 psi.

According to some working examples of the present disclosure, the predetermined ventilatory duration is characterized in having an inspiratory/expiratory (I/E) ratio of about 2:1 to 1:6.

Another aspect of the present disclosure is directed to a portable ventilator, which comprises a reservoir, at least two inlet flow valves, a frequency controller, a solenoid valve, an outlet flow meter and a control unit. In the structure, the reservoir is configured to house a gas mixture formed of pure oxygen and air, in which the gas mixture has a gas pressure; the at least two inlet flow valves are disposed upstream the reservoir and are configured to individually control the respective amount of the air and/or the pure oxygen in the gas mixture. The frequency controller is configured to apply a predetermined oscillatory frequency and a predetermined ventilatory duration to the gas mixture, thereby converts the gas mixture into the oscillatory flow. The solenoid valve is configured to output the oscillatory flow at a first jet pressure, in which the outputted oscillatory flow has a first flow rate. The outlet flow meter is disposed downstream the solenoid valve and configured to detect the first flow rate of the oscillatory flow. In addition, the control unit is configured to control the at least two inlet flow valves, the frequency controller, the solenoid valve and the outlet flow meter; the control unit is programmed with instructions to execute a method for modulating the oscillatory flow, the method comprises: (i) respectively matching the first flow rate and the first jet pressure with a predetermined flow rate and a predetermined jet pressure by varying the respective amounts of the pure oxygen and the air in the gas mixture; or (ii) matching the first jet pressure with the predetermined jet pressure by varying the predetermined ventilatory duration, in which if the fist jet pressure is smaller than the predetermined jet pressure, then decreases the predetermined ventilatory duration; or if the first jet pressure is greater than the predetermined jet pressure, then increases the predetermined ventilatory duration.

According to some embodiments of the present disclosure, the first jet pressure is substantially equal to the gas pressure and is in proportional to the amount of the gas mixture in the reservoir.

According to some embodiments of the present disclosure, the portable ventilator further comprises a pressure sensor coupled to the reservoir to detect the gas pressure of the gas mixture.

In certain embodiments, the pressure sensor is an absolute pressure sensor, a gauge pressure sensor, a vacuum pressure sensor, a differential pressure sensor, or a sealed pressure sensor.

According to some embodiments of the present disclosure, the at least one inlet flow valve controls the respective amount of the air and/or the pure oxygen in the gas mixture based on the gas pressure.

According to optional embodiments of the present disclosure, the portable ventilator further comprises an inlet flow meter disposed between the at least one inlet flow valve and the reservoir and configured to detect the respective flow rate of the air and the pure oxygen, thereby obtaining an actual oxygen concentration, wherein the inlet flow valve controls the amount of the pure oxygen in the gas mixture based on the actual oxygen concentration.

According to some embodiments of the present disclosure, the gas mixture has a predetermined oxygen concentration about 20 vol % to 90 vol %.

According to some embodiments of the present disclosure, the frequency controller comprises an oscillator and a digital-to-analog converter.

According to some optional embodiments of the present disclosure, the portable ventilator further comprises at least one check valve configured to respectively prevent the air, the pure oxygen and/or the oscillatory flow from flowing backwards.

In some optional embodiments, the portable ventilator further comprises an airway pressure sensor configured to determine an airway pressure of a subject.

According to certain embodiments of the present disclosure, the predetermined oscillatory frequency is about 1 Hz to 8 Hz, the predetermined jet pressure is about 5 psi to 45 psi, and the predetermined ventilatory duration is characterized in having an inhale/exhaled (I/E) ratio of about 2:1 to 1:6.

According to certain embodiments of the present disclosure, in the step (i) of the method, if the first flow rate is smaller than the predetermined flow rate, then increases the respective amounts of the pure oxygen and the air in the gas mixture; or if the first flow rate is greater than the predetermined flow rate, then decreases the respective amounts of the pure oxygen and the air in the gas mixture, wherein the predetermined flow rate is about 0 L/min to 30 L/min.

By virtue of the above configuration, the method of the present disclosure can regulate the portable ventilator of the present disclosure to adjust output airflow in real time as appropriate.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 is a flow chart illustrating a regulating method 1 for providing ventilation via a portable ventilator 2 according to the embodiment of the present disclosure;

FIG. 2 is a diagram illustrating the portable ventilator 2 according to the embodiment of the present disclosure;

FIG. 3 is a flow chart illustrating a regulating scheme according to the examples of the present disclosure; and

FIG. 4 depicts multiple integrated waveforms of an oscillatory flow produced by the portable ventilator 2 of the present disclosure; and

FIG. 5 depicts the results after a predetermined period of treatment by utilizing the present portable ventilator 2 with the regulating method 1.

In accordance with common practice, the various described features/elements are not drawn to scale but instead are drawn to best illustrate specific features/elements relevant to the present invention. Also, like reference numerals and designations in the various drawings are used to indicate like elements/parts.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about”. Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The term “ventilatory duration” used herein refers to the time durations of an expelled gas flow produced and outputted by the ventilator that basically complies with the inhalation and exhalation of a subject. The ventilatory duration can be controlled by opening or closing a valve near an outlet of the ventilator. The valve can be regulated based on preset signals, or can be adjusted based on any instant feedback. In the practical operation of the ventilator of the present disclosure, the ventilatory duration substantially represents an inspiratory duty cycle, or, a ratio of the inspiratory duration to the expiratory duration (I/E ratio).

The term “subject” or “patient” is used interchangeably herein and is intended to refer a mammal including Homo species that is subjectable to the ventilating device and/or the regulation method thereof of the present invention. The term “mammal” refers to all members of the class Mammalia, including human races (Homo sapiens), primates, domestic and farm animals, such as rabbit, pig, sheep, and cattle; as well as zoo, sports or pet animals. Further, the term “subject” or “patient” intended to refer to both the male and female gender unless one gender is specifically indicated. Accordingly, the term “subject” or “patient” comprises any mammal which may benefit from the treatment method of the present disclosure. Examples of a “subject” or “patient” include, but are not limited to, a human, monkey, pig, goat, cow, horse, dog, cat, and etc. In some exemplary embodiments, the subject is a pig or a human.

### II. Description of the Invention

The invention aims at providing a portable respiratory apparatus and an improved method for providing high-frequency ventilation (e.g., an oscillatory flow) to a subject in need thereof. Particularly, the oscillatory flow produced from the respiratory apparatus is modulated by the present method.

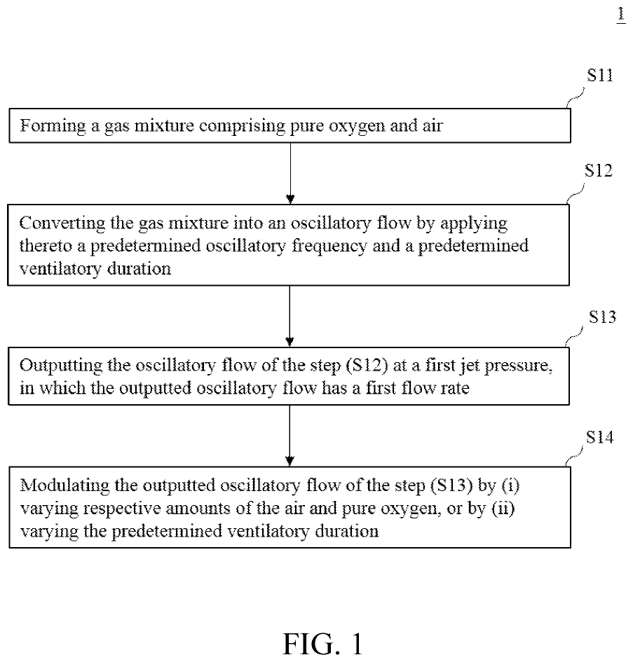

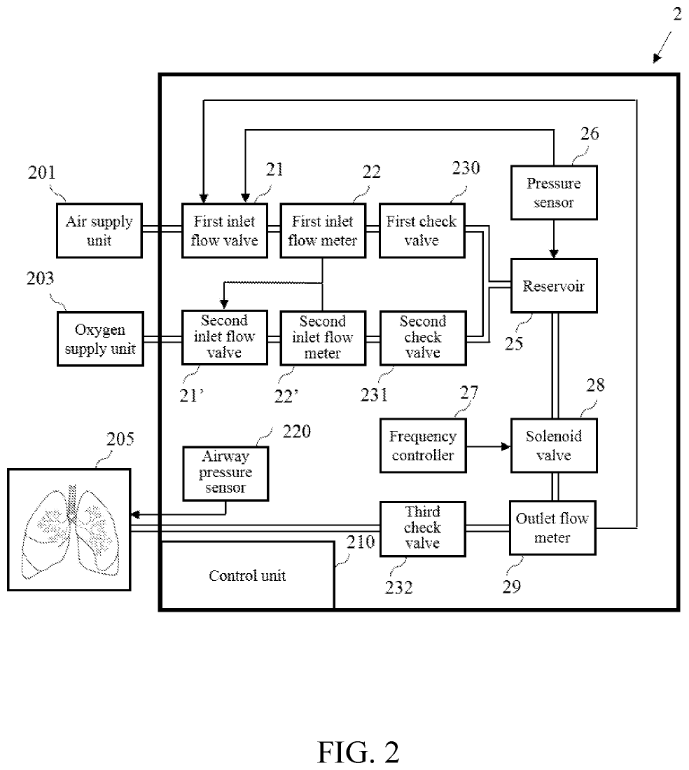

Description is now directed to embodiments of the present invention with references made to both FIGS. 1 and 2. FIG. 1 is a flow chart depicting the steps of the present regulating method 1, and FIG. 2 is a schematic drawing of a portable ventilator 2 for implementing the present method. In practice, the portable ventilator 2 is coupled to an air supply unit 201 and a pure oxygen supply unit 203 respectively containing cylinders for providing gas to the portable ventilator 2, the gas is subsequently outputted from the portable ventilator 2 as an oscillatory flow, preferably being modulated by the present method, to the lungs of a subject 205.

In the present method, ventilation starts by first inputting air and pure oxygen to a reservoir 25 in the portable ventilator 2 to form a gas mixture (S11). The air and the pure oxygen are independently supplied from the air supply unit 201 and the pure oxygen supply unit 203 with the aid of cylinders, which are independently set to provide air and pure oxygen at a predetermined pressure, such as at the pressure of 50 psi.

The gas mixture thus formed in the step S11 is then converted and outputted as an oscillatory flow (S12 and S13). To this purpose, a predetermined oscillatory frequency and a predetermined ventilatory duration are applied to the gas mixture, so that it may be outputted in the form of an oscillatory flow via a jet pressure. During operation, as air and oxygen continue to enter the reservoir 25 (S11), pressure in the portable reservoir 25 therefore continues to increase in proportional to the number of total gas molecules therein for the volume of the reservoir 25 in the ventilator 2 remains constant in the present invention, thus the gas mixture housed therein behaves in a manner that complies with the ideal gas law, where the pressure of the gas mixture is in proportional to the number of total gas molecules housed in the reservoir 25. This pressure, generated by the accumulated gas molecules in the reservoir 25, is termed “jet pressure” in the present disclosure. The jet pressure also serves as the driving force for discharging or outputting the gas mixture housed therein through a solenoid valve 28. In some embodiments, the jet pressure is below the pressure set for the air supply unit 201 and the pure oxygen supply unit 203. In some embodiments, the jet pressure is not greater than 45 psi. In other embodiments, the jet pressure is from 5 psi to 45 psi, for example, 5, 10, 15, 20, 25, 30, 35, 40, or 45 psi. In preferred embodiments, the jet pressure is from 15 psi to 25 psi.

To produce an oscillatory flow, a predetermined oscillatory frequency and a predetermined ventilatory duration are applied to the gas mixture by a frequency controller 27 to convert the gas mixture into an oscillatory flow before being outputted from the solenoid valve 28 at the jet pressure (referring to S13). The frequency controller 27 is configured to generate oscillatory frequency and ventilatory duration based on commands of a user inputted from a control panel in the control unit 210, such as inspiratory duty cycle (i.e., inspiratory/expiratory (I/E) ratio). According to some embodiments, the frequency controller 27 comprises an oscillator, a digital-to-analog converter, microprocessors, and a comparator, in which the oscillator and the digital-to-analog converter are both subjected to digital signals derived from the microprocessors and are connected to the comparator to generate an integrated waveform containing information of the oscillatory frequency and the ventilatory duration (e.g., inspiratory duty cycle). The microprocessors are configured to respectively control the oscillator and the digital-to-analog converter via regulating capacitance value and voltage value based on said digital signals. Typically, the oscillator is configured to produce a periodic, oscillating electronic signal (often a sine wave and/or a square wave) to the comparator, thereby outputs a frequency signal to a certain device (e.g., the solenoid valve 28 in the present portable ventilator 2) for further usage. In addition, the oscillator can produce various frequency of the output signal, for instance, from below 1 Hz to over 100 kHz. In some embodiments, the present oscillator produces signal with the frequency below 20 Hz, for example, from 1 Hz to 15 Hz, from 1 Hz to 10 Hz, from 1 Hz to 8 Hz, from 1 Hz to 5 Hz, from 2 Hz to 4 Hz, or from 1 Hz to 3 Hz. In some embodiments, the oscillator is designed so that the oscillation frequency can be varied over some range by an input voltage or current. In the preferred embodiment, the oscillator is a voltage-controlled oscillator (VCO) that provides an adjustable oscillatory frequency based on practical needs.

To achieve optimal ventilation, the oscillatory flow needs to be fine-tuned or modulated so that the flow rate and/or the jet pressure match with parameters preset by the user. Examples of the preset parameters include, but are not limited to, flow rates and/or concentrations of air and pure oxygen, jet pressure, oscillatory frequency, ventilatory duration (e.g., I/E ratio) and etc. These preset parameters may be directly inputted from the control unit 210 by the user before starting the ventilation.

According to embodiments of the present disclosure, the control unit 210 can execute the modulating method via programmed instructions including or other than the parameters above-mentioned. Specifically, in accordance with the method executed by the control unit 210, the oscillatory flow outputted at the jet pressure may be modulated by varying respective amounts of the air and pure oxygen, which in terms varying the jet pressure as well as the flow rate of the oscillatory flow; or by varying the predetermined ventilatory duration (S14). Additionally or alternatively, the oscillatory flow in S13 may be further modulated by varying the concentration of pure oxygen that enters the ventilator 2. Accordingly, the main route to modulate the oscillatory flow is through flow rate control, which monitors the flow rate and the jet pressure of the oscillatory flow. Additionally or alternatively, the oscillatory flow is modulated through concentration control, which monitors and varies the concentration of the pure oxygen in the gas mixture.

(i) Flow Rate Control Route

The flow rate of the oscillatory flow may be determined by an outlet flow meter 29 disposed downstream of the solenoid valve 28, and is subsequently compared with a predetermined flow rate. The predetermined flow rate may be a value predetermined by a user; alternatively, it may be derived from the respective flow rates of pure oxygen and air that enter the portable ventilator 2. In the case when the flow rate of the oscillatory flow determined by the outlet flow meter 29 is smaller than the predetermined flow rate, then the first and second inlet flow valves (21, 21′) are opened to allow more air and pure oxygen entering the portable ventilator 2, so as to increase the detected flow rate until it matches with the predetermined flow rate. Note that the respective flow rates of air and pure oxygen are determined by the first and second inlet flow meters (22, 22′), which are, in the present embodiment, disposed downstream the first and second inlet flow valves (21, 21′). On the other hand, in the case when the determined flow rate is greater than the predetermined flow rate, then the first and second inlet flow valves (21, 21′) are closed to allow less air and pure oxygen entering the portable ventilator 2, so as to decrease the detected flow rate until it is reduced to the predetermined flow rate. According to embodiments of the present disclosure, the predetermined flow rate may be in the range of about 0.5 L/min to 30 L/min, such as 0.5, 1, 5, 10, 25, 20, 25 and 30 L/min. In one preferred embodiment, the predetermined flow rate is about 25 L/min. Note that the first and second inlet flow valves (21, 21′) may be any valve that regulates, directs or controls the flow of gases by opening, closing, or partially obstructing various passageways. In certain embodiments, the first and second inlet flow valves (21, 21′) are proportional control valves independently having the ability to control the position of the internal spool assembly that increases or decreases the amount of flow being released from the valve. In preferred embodiment, the first and second inlet flow valve (21, 21′) are independently electro-pneumatic proportional valves that can be controlled via voltage. Accordingly, the flow rate of the air or the pure oxygen are adjusted by opening or closing the first or second inlet valves (21, 21′) to increase or decrease the amounts of air or pure oxygen entering the portable ventilator 2 according to practical needs. Further, based on the flow rates of the air and the pure oxygen respectively determined by the first and second inlet flow meters (22, 22′), the oxygen concentration in the gas mixture may be derived and regulated in real-time.

Alternatively, the modulation is achieved via monitoring the jet pressure, which is determined by a pressure sensor 26 coupled to the reservoir 25. Additionally, the pressure sensor 26 also serves as a transducer that regulates the first and second inlet flow valves (21, 21′), thus, the jet pressure may be matched with a predetermined jet pressure value (e.g., a value preset by a user) by opening or closing the first and second inlet flow valves (21, 21′) to allow more or less amounts of air and pure oxygen to enter the portable ventilator 2. Examples of the pressure sensor 26 include, but are not limited to, an absolute pressure sensor, a gauge pressure sensor, a vacuum pressure sensor, a differential pressure sensor, and a sealed pressure sensor. In the preferred embodiment, the pressure sensor 16 is gauge pressure sensor.

Alternatively, or in addition, in certain embodiments, the jet pressure may be matched with the predetermined jet pressure value by altering the ventilatory duration, instead of by varying the respective flow rates of pure oxygen and air that enter the portable ventilator 2. In such case, if the jet pressure is smaller than the predetermined jet pressure value, then decreases the predetermined ventilator duration (e.g., by decreasing the I/E ratio); on the other hand, if the jet pressure is greater than the predetermined jet pressure value, then increases the predetermined ventilator duration (e.g., by increasing I/E ratio). According to embodiments of the present disclosure, the I/E ratio is in the range of about 2:1 to 1:6, such as 2:1, 1.5:1, 1:1, 1:1.5, 1:2, 1:2.5, 1:3, 1:3.5, 1:4, 1:4.5, 1:5, 1:5.5 and 1:6; preferably about 1:1 to 1:4; and more preferably, about 1:2.

(ii) Concentration Control Route

Another approach for modulating the oscillatory flow is through varying the concentration of the pure oxygen in the gas mixture. To this purpose, the actual concentration of pure oxygen in the gas mixture is determined and matched with a predetermined oxygen concentration, which in general, falls in the range of 20-90% by volume, such as 20, 25, 30, 35, 40, 45, 50, 55, 60, 65, 70, 75, 80, 85 and 90% by volume (vol %). In one preferred embodiment, the predetermined oxygen concentration is 20%. In some embodiments, the actual concentration of pure oxygen in the gas mixture is derived from the flow rate of pure oxygen determined by the second inlet flow meter 22′. In the case when the actual oxygen concentration is lower than the predetermined value, the second inlet flow valve 21′ is opened to allow more pure oxygen to enter the portable ventilator 2. On the other hand, if the actual oxygen concentration is greater than the predetermined value, then the second inlet flow valve 21′ is closed to allow less pure oxygen to enter the portable ventilator 2. Additionally or optionally, the actual concentration of pure oxygen is determined prior to, or after the output of the oscillatory flow.

By virtue of the afore-mentioned manners, optimal ventilation may be achieved by outputting the gas mixture in a desired oscillatory flow at conditions that match preset parameters (e.g., jet pressure, I/E ratio, and etc).

Additionally, or optionally, the present method further includes the step of determining the airway pressure of the subject 205 (e.g., via use of an airway pressure sensor 220) to facilitate real-time monitoring and/or adjusting the jet pressure, and the oscillatory flow. Preferably, the monitoring results is displayed on the screen of the user interface (e.g., the control unit 210). Additionally, or optionally, the control unit 210 may be further configured to send an alarm if the airway pressure of the patient fails to match with the predetermined value.

Additionally, or optionally, the present invention also includes means to ensure the gas in the portable ventilator 2, which may be the air, the pure oxygen and the gas mixture, may only flow in one direction. To this purpose, a plurality of check valves are disposed upstream and/or downstream of the reservoir 25 to prevent gas from flowing backwards. According to preferred embodiments, three check valves 230, 231, and 232 are respectively disposed in the portable ventilator 2. The first check valve 230 is disposed between the first inlet flow meter 22 and the reservoir 25; the second check valve 231 is disposed between the second inlet flow meter 22′ and the reservoir 25; and the third check valve 232 is disposed downstream the outlet flow meter 29. Specifically, the first check valve 230 and the second check valve 231 are respectively configured to prevent the air and the pure oxygen flow reversely back toward the air supply unit 201 and the pure oxygen supply unit 203. The third check valve 232 is configured to prevent the oscillatory flow pass countercurrent to the outlet flow meter 29.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

Materials and Methods

Animals

Six miniature pigs (Landrace pig strain name, about 40 Kg) were obtained from a private farm (Bali District, New Taipei City)) and maintained in AAALAC-accredited laboratory animal facility with experimental procedures for handling the pigs complied with relevant regulations set forth in “Guide for the Care and Use of Laboratory Animals: Eighth Edition” (National Academies Press, Washington, D.C., 2011).

Acute Respiratory Distress-Like (ARDL) Syndrome and Ventilation Treatment

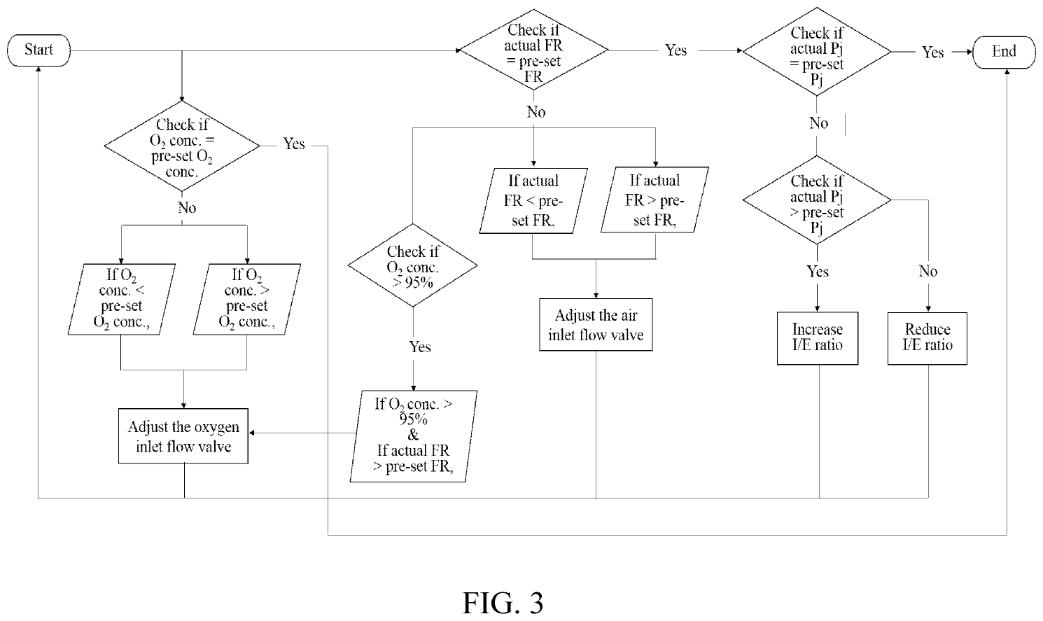

In this example, animals were first subjected to normal ventilation, then were given oleic acid to induce acute respiratory distress-like (ARDL) syndrome. Animals exhibiting ARDL syndrome (i.e., PaO₂<60 mmHg) were then treated with the high frequency ventilation generated by the ventilator of Example 1 in accordance with a regulatory scheme depicted in FIG. 3. During ventilation, arterial blood gas (ABG) data were collected every 10 mins for evaluation of the treatment.

Specifically, ventilation was administered to each animal via endotracheal intubation, in which endotracheal tube (2 mm in diameter) was inserted into the trachea until it was about 3-4 cm above the carina. Normal ventilation was started by setting the tidal volume (Vt) to be 10 mL/kg, and the respiratory rate (RR) to be 15/min. Then, each pig was given 0.2 ml of oleic acid 50% (diluted in methanol 95%) every 2 minutes to induce acute respiratory distress-like (ARDL) syndrome, which was characterized in having the partial pressure of oxygen (PaO₂) being smaller than 60 mmHg.

Animals exhibiting ARDL syndrome (i.e., PaO₂<60 mmHg) were connected to the portable ventilator of Example 1, and high-frequency ventilation generated in accordance with the regulating scheme depicted in FIG. 3 was applied thereto for 30 minutes. Before starting the ventilation, preset parameters were entered by the user, which included, at least, flow rate of the oscillatory flow (denoted as “FR” in FIG. 3), jet pressure (denoted as “Pⱼ” in FIG. 3), oxygen concentration (denoted as “O₂ conc.” in FIG. 3) in the gas mixture, I/E ratio, and oscillatory frequency. The ventilator was allowed to run for a few minutes based on the preset values, then shifted to the regulating scheme depicted in FIG. 3, in which the actual flow rate of the oscillatory flow, the actual jet pressure and the actual oxygen concentration were respectively determined and adjusted via varying the respective flow rates of air and pure oxygen that entered the ventilator, or via varying the I/E ratio. Animals were sacrificed at the end of the experiment, and their lung tissues were collected for subsequent microscopic examination.

### Example 1 Construction of the Present Portable Ventilator

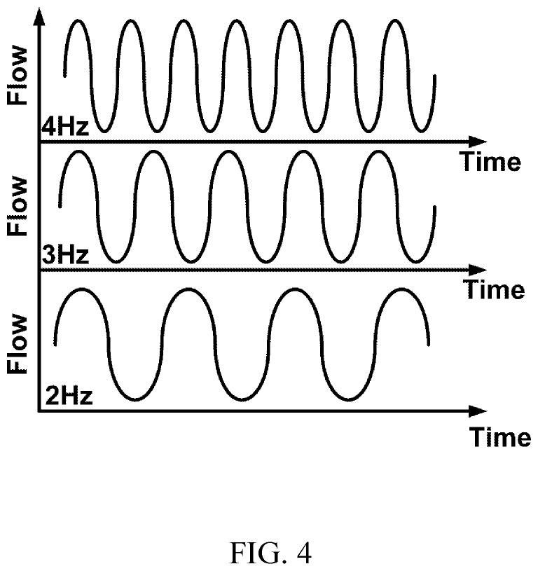

All components of the present ventilator were respectively purchased from commercial sources and arranged substantially in accordance with the layout depicted in FIG. 2, which was configured to implement the regulatory scheme of FIG. 3 to generate high frequency ventilation of the present invention. In addition, the integrated waveform of the produced oscillatory flow by the portable ventilator 2 at various frequencies are depicted in FIG. 4. The size of the ventilator thus constructed was about 20 cm/15 cm /17 cm (length/width/height) in size.

### Example 2 Treating ADRL Subjects with High Frequency Ventilation Generated by the Portable Ventilator of Example 1

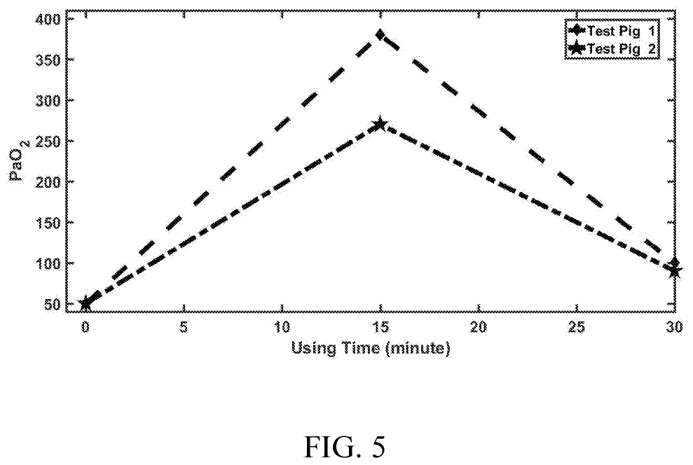

In the present example, miniature pigs exhibiting ADRL syndrome were treated with high frequency ventilation generated by the ventilator of Example 1, in which the ventilator was first run for a few minutes based on preset parameters, then it was shifted to perform the regulatory scheme of FIG. 3 for 15 minutes. Results are summarized in FIG. 5, in which each line represents each tested individual. The preset parameters included: tidal volume (Vt) of 3 mL/kg, respiratory rate (RR) of 120/min, jet pressure of 20 psi, and fraction of inspired oxygen (FiO₂, or the O₂ concentration) of 1 (i.e., 100% oxygen).

As shown in FIG. 5, the measured PaO₂ at the beginning of the ventilation (time=0) was smaller than 60 mmHg, indicating the subject had ARDL syndrome. After administering ventilation (which was generated based on preset values) for 15 minutes, the regulating scheme of FIG. 3 was performed based on the collected ABG data. By regulating the jet pressure and the I/E ratio via varying the respective flow rate of air and pure oxygen, the ventilatory frequency and the inspiratory volume were adjusted into a normal range (PaO₂=80-100 mmHg) within the next 15 minutes.

In addition, microscopy examination of the lung tissue of the tested subjects confirmed that ventilation provided by the present ventilator of Example 1 could rescue the damage lung tissue (data not shown).

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method for providing an oscillatory flow to a subject in need thereof, comprising,
(a) forming a gas mixture comprising pure oxygen and air;
(b) converting the gas mixture into the oscillatory flow by applying thereto a predetermined oscillatory frequency and a predetermined ventilatory duration;
(c) outputting the oscillatory flow of the step (b) at a first jet pressure, in which the outputted oscillatory flow has a first flow rate; and
(d) modulating the outputted oscillatory flow of the step (c) by,
(i) respectively matching the first flow rate and the first jet pressure with a predetermined flow rate and a predetermined jet pressure by varying the respective amounts of the pure oxygen and the air in the gas mixture; or
(ii) matching the first jet pressure with the predetermined jet pressure by varying the predetermined ventilatory duration of the step (b), in which
if the fist jet pressure is smaller than the predetermined jet pressure, then decreases the predetermined ventilatory duration; or
if the first jet pressure is greater than the predetermined jet pressure, then increases the predetermined ventilatory duration.

2. The method of claim 1, wherein in the step (a), the pure oxygen and the air are independently suppled from their sources and mixed in a reservoir having a constant volume to form the gas mixture.

3. The method of claim 2, wherein in the step (c), the first jet pressure of the oscillatory flow is in proportional to the amount of the gas mixture in the reservoir.

4. The method of claim 1, wherein in the step (d)(i),
if the first flow rate is smaller than the predetermined flow rate, then increases the respective amounts of the pure oxygen and the air in the gas mixture; or
if the first flow rate is greater than the predetermined flow rate, then decreases the respective amounts of the pure oxygen and the air in the gas mixture.

5. The method of claim 1, wherein the predetermined flow rate is about 0 L/min to 30 L/min.

6. The method of claim 1, wherein the gas mixture of the step (a) has a predetermined oxygen concentration.

7. The method of claim 6, further comprising,
(e) detecting an actual oxygen concentration in the gas mixture in the step (c) and/or after the step (d)(i); and
(f) matching the actual oxygen concentration with the predetermined oxygen concentration by varying the amount of the pure oxygen in the gas mixture of the step (a).

8. The method of claim 7, wherein the predetermined oxygen concentration is about 20% to 90%.

9. The method of claim 1, wherein the predetermined oscillatory frequency is about 1 Hz to 8 Hz, and the predetermined jet pressure is about 5 psi to 45 psi.

10. The method of claim 1, wherein the predetermined ventilatory duration is characterized in having an inhale/exhaled (UE) ratio of about 2:1 to 1:6.

11. A portable ventilator for providing an oscillatory flow, comprising,
a reservoir configured to house a gas mixture formed of pure oxygen and air, in which the gas mixture has a gas pressure;
at least two inlet flow valves disposed upstream the reservoir and configured to individually control the respective amount of the air and/or the pure oxygen in the gas mixture;
a frequency controller configured to apply a predetermined oscillatory frequency and a predetermined ventilatory duration to the gas mixture, thereby converts the gas mixture into the oscillatory flow;
a solenoid valve configured to output the oscillatory flow at a first jet pressure, in which the outputted oscillatory flow has a first flow rate;
an outlet flow meter disposed downstream the solenoid valve and configured to detect the first flow rate of the oscillatory flow; and
a control unit configured to control the at least two inlet flow valves, the frequency controller, the solenoid valve and the outlet flow meter, wherein the control unit is programmed with instructions to execute a method for modulating the oscillatory flow, comprising,
(i) respectively matching the first flow rate and the first jet pressure with a predetermined flow rate and a predetermined jet pressure by varying the respective amounts of the pure oxygen and the air in the gas mixture; or
(ii) matching the first jet pressure with the predetermined jet pressure by varying the predetermined ventilatory duration, in which
if the fist jet pressure is smaller than the predetermined jet pressure, then decreases the predetermined ventilatory duration; or
if the first jet pressure is greater than the predetermined jet pressure, then increases the predetermined ventilatory duration.

12. The portable ventilator of claim 11, wherein the first jet pressure is substantially equal to the gas pressure and is in proportional to the amount of the gas mixture in the reservoir.

13. The portable ventilator of claim 11, further comprising a pressure sensor coupled to the reservoir to detect the gas pressure of the gas mixture.

14. The portable ventilator of claim 13, wherein the pressure sensor is an absolute pressure sensor, a gauge pressure sensor, a vacuum pressure sensor, a differential pressure sensor, or a sealed pressure sensor.

15. The portable ventilator of claim 13, wherein the at least one inlet flow valve controls the respective amount of the air and/or the pure oxygen in the gas mixture based on the gas pressure.

16. The portable ventilator of claim 11, wherein the at least one inlet flow valve controls the respective amount of the air and/or the pure oxygen in the gas mixture based on the first flow rate.

17. The portable ventilator of claim 11, further comprising an inlet flow meter disposed between the at least one inlet flow valve and the reservoir and configured to detect the respective flow rate of the air and the pure oxygen, thereby obtaining an actual oxygen concentration, wherein the inlet flow valve controls the amount of the pure oxygen in the gas mixture based on the actual oxygen concentration.

18. The portable ventilator of claim 11, wherein the gas mixture has a predetermined oxygen concentration about 20 vol % to 90 vol %.

19. The portable ventilator of claim 11, wherein the frequency controller comprises an oscillator and a digital-to-analog converter.

20. The portable ventilator of claim 11, further comprising at least one check valve configured to respectively prevent the air, the pure oxygen and/or the oscillatory flow from flowing backwards.

21. The portable ventilator of claim 11, further comprising an airway pressure sensor configured to determine an airway pressure of a subject.

22. The portable ventilator of claim 11, wherein the predetermined oscillatory frequency is about 1 Hz to 8 Hz, the predetermined jet pressure is about 5 psi to 45 psi, and the predetermined ventilatory duration is characterized in having an inhale/exhaled (UE) ratio of about 2:1 to 1:6.

23. The portable ventilator of claim 11, wherein in the step (i) of the method,
if the first flow rate is smaller than the predetermined flow rate, then increases the respective amounts of the pure oxygen and the air in the gas mixture; or
if the first flow rate is greater than the predetermined flow rate, then decreases the respective amounts of the pure oxygen and the air in the gas mixture,
wherein the predetermined flow rate is about 0 L/min to 30 L/min.
